(19) United States
(12) Patent Application Publication
(10) Pub. No.: US 2010/0310486 A1
(43) Pub. Date: Dec. 9, 2010

(54) COSMETIC COMPOSITION COMPRISING MACADAMIA OIL, AND A WAX

(75) Inventors: Xavier Blin, Paris (FR); Sylvie Guillard, Chevry Cossigny (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 12/747,775

(22) PCT Filed: Dec. 11, 2008
(86) PCT No.: PCT/IB08/55227

§ 371 Date: Aug. 17, 2010

Related U.S. Application Data
(60) Provisional application No. 61/006,266, filed on Jan. 3, 2008.

(30) Foreign Application Priority Data
Jan. 3, 2008 (FR) 0759805

(51) Int. Cl. A61K 8/92; A61Q 1/06

(52) U.S. Cl. 424/64

(57) ABSTRACT

The present invention relates to an anhydrous cosmetic composition for making up and/or caring for the lips comprising at least one fatty phase comprising at least 0.5% by weight, with respect to the total weight of the composition, of at least one macadamia oil or one of its derivatives and at least one polyglycerolated beeswax.

The present invention relates to a makeup and/or care cosmetic composition, in particular for the lips.

A lipstick is today an inescapable makeup product which, beyond its mission of making up the lips, also constitutes a care product or a balm for the lips for numerous users suffering from dryness of the lips.

It is therefore very important for the technical properties of a lipstick, such as the gloss, the hold and the colouring quality, to be combined with qualities of care and of comfort, this being the case on application but also over time.

These complex properties can be adjusted via the presence in these lipsticks of certain active principles but, for obvious reasons, the latter cannot satisfy all the expectations on the part of users and in particular the request for comfort, which property concerns a multisensorial sensation resulting from individual perceptions on application and from the wearing of the film over time.

Furthermore, for lipsticks of this type, the detailed expectations of the user, who is increasingly looking for products having constituents of natural origin, cannot be ignored.

This request, which corresponds to a current expectation but one which will probably be lasting, can be satisfied by the use of new ingredients described as natural which are satisfactory with regard to the formulation but which may, on the other hand, for some of them, cause additional problems from the viewpoint of their organoleptic qualities.

In point of fact, in the case of lipsticks, these organoleptic characteristics (such as smell and taste problems) are essential and cannot be neglected.

In addition, these “natural” starting materials very often exhibit a change over time such that the organoleptic properties and/or their quality are generally affected. This is because it is frequent for these formulations to be confronted with stability problems which are, of course, undesirable.

It is a specific subject-matter of the present invention to satisfy all the expectations specified above.

The present invention results more particularly from the unexpected observation by the inventors that a specific combination of “natural” fatty substances makes it possible, on the one hand, to meet the expectations of users in terms of comfort and, on the other hand, to be spared the organoleptic failings encountered with conventional natural products.

Thus, according to a first of its aspects, the present invention relates to an anhydrous cosmetic composition for making up and/or caring for the lips comprising a fatty phase comprising:
at least 0.5% by weight of at least one macadamia oil or one of its derivatives, and
at least one polyglycerolated beeswax.

The inventors have thus found that a composition comprising a macadamia oil, which does not present an organoleptic problem in contrast to a large number of natural oils, or one of its derivatives, in combination with an animal or vegetable wax, such as, in particular, polyglycerolated beeswax, has particularly satisfactory properties in terms of stability (lack of wax recrystallisation, particularly on the surface of the stick).

As established by inventors, an association of one Macadamia oil with such a wax allows to reinforce the stability of the composition containing it. Thus, no phenomenon of exudation and/or of recrystallisation is established over time.

Finally, the deposited layers of makeup obtained from the application of these compositions in accordance with the invention to the keratinous substance, in this case the lips, indicate satisfactory properties of comfort, both on application and during wearing over time (after one or several hours). Thus, the compositions of the invention prove to be highly satisfactory in terms of ease of application (slip and disintegration).

According to a first embodiment, the composition according to the invention is provided in the form of products cast as a stick or in a dish and is then described as solid.

According to a specific embodiment, the composition according to the invention can be provided in the form of a lipstick stick.

The term “solid” characterizes the state of the composition at ambient temperature (25° C.) and at atmospheric pressure (760 mmHg).

Thus, a solid composition can advantageously exhibit a hardness varying from 30 to 400 g, in particular from 50 to 300 g, indeed even from 80 to 250 g.

This hardness can in particular be measured according to the following protocol, also known as “cheesewire method”.

A sample of the composition under consideration is hot cast in a mould for sticks with a diameter of 12.7 mm. The mould is subsequently cooled in a freezer for approximately one hour. The lipstick stick is subsequently stored at 20° C.

The hardness of the samples is measured after waiting for 24 hours.

The hardness of the samples of compositions of the invention, expressed in grams, is measured using a DFGS2 tensile testing device sold by Indelco-Chatillon.

The hardness corresponds to the maximum shear force exerted by a stiff tungsten wire with a diameter of 250 μm advancing at a rate of 100 mm/min.

According to an alternative embodiment, a composition according to the invention can be provided in a fluid form, for example of gloss type.

Within the meaning of the invention, the term “anhydrous” means that the said composition comprises less than 4% by weight of water, and in particular less than 2% by weight of water, with respect to the total weight of the composition, indeed is even entirely devoid of water.

Preferably, water is not added during the preparation of the composition, but is corresponding to residual water brought by mixed ingredients.

According to another aspect, the present invention relates to a method for making up or caring for the lips, comprising at least one stage which consists in applying, to the said lips, a composition as defined above.

Macadamia Oil or Derivatives

Macadamia oil is an oil extracted from macadamia nuts.

Within the meaning of the present invention, the term “macadamia nut oil derivative” is understood to mean either products obtained on conclusion of specific treatments applied to macadamia oil or a compound or a mixture of compounds capable of being extracted from this oil.

Thus, various treatments are capable of being applied to this macadamia nut oil, in particular so as to formulate it with different degrees of refining, for example. Thus, in the context of the present invention, the macadamia nut oil can be employed in a crude form, in a deodorized form or else in a refined form.

Mention may in particular be made, by way of representation of the extracts of macadamia oil, of the fatty acid triglycerides of which it is composed and their mixtures. Palmitic acid, palmitoleic acid, stearic acid, oleic acid and linoleic acid are involved in particular.

Thus, a macadamia oil derivative can be symbolized by a mixture of triglycerides of palmitoleic/oleic acid in variable ratios. Mention may in particular be made, by way of representation of such a mixture, of that sold by Milor Colaborative DGT and formed of a 25/55 mixture of palmitoleic/oleic acid.

The derivatives within the meaning of the present invention also cover the hydrogenated forms of macadamia oil. These derivatives can also be ethyl esters of macadamia oil or also ethoxylated forms of macadamia oil.

Finally, the macadamia oil or one of its derivatives, as defined above, can be employed in a pure form but also in the form of a mixture.

Numerous mixtures of macadamia oil are thus available commercially.

Mention may particularly be made, by way of representation of these mixtures, of those in which the macadamia oil is combined with coriander oil, jojoba oil, passionflower oil, apricot kernel oil, borage oil and wheat germ oil.

For obvious reasons, only the mixtures or derivatives capable of reproducing the properties displayed by macadamia oil will be considered in the context of the present invention.

As specified above, the cosmetic compositions according to the present invention comprise at least 0.5% by weight of macadamia oil or derivatives, with respect to their total weight.

More particularly, these compositions can comprise at least 2% by weight, in particular at least 3% by weight, especially at least 5% by weight, especially at least 7% by weight and more particularly at least 10% by weight, indeed even at least 20% by weight, of macadamia oil or derivatives, with respect to their total weight.

More particularly, these compositions may contain between 2% and 60% by weight, in particular between 3% and 60%, especially between 5% and 50%, especially between 7% and 50% and more especially between 10% and 50% by weight, particularly between 20% and 45% by weight of macadamia oil or derivatives thereof with respect to their total weight.

Polyglycerolated Beeswax

Generally, a wax considered in the context of the present invention is a lipophilic compound which is solid at ambient temperature (25° C.), which exhibits a reversible solid/liquid change in state and which has a melting point of greater than or equal to 30° C., which can range up to 200° C. and in particular up to 120° C.

In particular, the waxes suitable for the invention can exhibit a melting point of greater than or equal to 45° C. and in particular of greater than or equal to 55° C.

Within the meaning of the invention, the melting point corresponds to the temperature of the most endothermic peak observed by thermal analysis (DSC: Differential Scanning Calorimetry) as described in Standard ISO 11357-3; 1999. The melting point of the wax can be measured using a differential scanning calorimeter, for example the calorimeter sold under the name “MDSC 2920” by TA Instruments.

The measurement protocol is as follows:

A 5 mg sample of wax placed in a crucible is subjected to a first rise in temperature ranging from −20° C. to 100° C. at a heating rate of 10° C./minute, is then cooled from 100° C. to −20° C. at a cooling rate of 10° C./minute and, finally, is subjected to a second rise in temperature ranging from −20° C. to 100° C. at a heating rate of 5° C./minute. During the second rise in temperature, the variation in the difference in power absorbed by the empty crucible and by the crucible comprising the sample of wax is measured as a function of the temperature. The melting point of the compound is the value of the temperature corresponding to the tip of the peak of the curve representing the variation in the difference in power absorbed as a function of the temperature.

As specified above, the compositions according to the invention comprise at least one polyglycerolated beeswax which is solid at ambient temperature.

Within the meaning of the invention, the terms “polyglycerolated beeswax” mean a beeswax of which fat acids are esterified with polyglycerid compounds.

In particular, a beeswax according to the invention is the polyglycerol-3-beeswax (beeswax esterified with polyglycerid-3), also identified under the name INCI: polyglycerid-3 beeswax, sold under the trademark “CERA BELLINA®” by KOSTER KEUNEN, and their mixtures. Such a wax is in particular disclosed in the U.S. Pat. No. 4,948,584.

According to a preferred embodiment, the polyglycerolated beeswax used in the composition of the invention is modified polyglycerol-3-beeswax.

Generally, the wax or waxes of natural origin can be present in a proportion of at least 2% by weight, in particular of at least 3% by weight, in particular of at least 4% by weight, in particular of at least 5% by weight, with respect to the weight of the fatty phase of the compositions according to the invention.

In particular, the polyglycerolated beeswax can be present in a proportion of at least 2 to 25%, especially from 3 to 25%, more especially from 4 to 25%, and in particular from 5 to 20% by weight with respect to the weight of the fatty phase of the compositions according to the invention.

A composition according to the invention can comprise from 2 to 15% by weight of wax(es) of natural origin, in particular at least 3% by weight, indeed even at least 5% by weight, of wax(es) of natural origin.

In particular, a composition according to the invention can comprise from 2 to 15% by weight of polyglycerolated beeswax, in particular from 3 to 13% by weight, in particular from 5 to 13% by weight of polyglycerolated beeswax.

According to an advantageous embodiment, a cosmetic composition according to the invention can combine a macadamia oil, in particular in a proportion of at least 15% by weight, with at least one wax of natural origin and more particularly a polyglycerolated beeswax, and more particularly from 15% to 50% by weight with at least one polyglycerolated beeswax.

According to an alternative embodiment, this wax can be present in a proportion of at least 5% by weight, with respect to the total weight of the composition.

Ester Oil

The inventors have also found that it is advantageous to formulate a composition according to the invention with at least one further ester oil. The corresponding compositions prove to be particularly advantageous for their properties in terms of comfort.

A composition according to the invention may in particular comprise at least 5% by weight, especially at least 10%, in particular at least 15%, in particular at least 18% and more particularly at least 20% of ester oil(s) with respect to the total weight of the composition.

In particular, a composition according to the invention may in particular contain from 5% to 60% by weight, in particular from 10% to 55%, in particular from 15% to 55%, in particular from 18% to 55% and more particularly from 20% to 50% of ester oil(s) with respect to the total weight of the composition.

A composition of the invention can comprise at least one additional ester oil chosen from linear ester oils and branched ester oils.

According to a preferred embodiment, the composition of the invention comprises at least one linear ester oil.

This linear ester oil is of formula R₁COOR₂ in which R₁ represents a residue of a linear or branched fatty acid comprising from 1 to 40 carbon atoms, and R₂ represents a hydrocarbon chain, in particular a branched hydrocarbon chain, comprising from 1 to 40 carbon atoms, provided that R₁+R₂≧10.

According to a preferred embodiment, the composition comprises at least one ester oil chosen from esters of alcohol and of fatty acid, such as, for example, cetostearyl octanoate, esters of isopropyl alcohol, such as isopropyl myristate or isopropyl palmitate, ethyl palmitate, 2-ethylhexyl palmitate, isopropyl stearate or isostearate, isostearyl isostearate, octyl stearate, octyldodecyl stearate, pentaerythrityl isostearate or caprate or caprylate or their mixtures, such as “Supermol L®” from CRODA (mixtures of pentaerythrityl isosterate/caprylate/adipate), hydroxylated esters, such as isostearyl lactate or octyl hydroxystearate, diisopropyl adipate, heptanoates and in particular isostearyl heptanoate, octanoates, decanoates or ricinoleates of alcohols or of polyalcohols, such as propylene glycol dioctanoate, cetyl octanoate, tridecyl octanoate, 4-diheptanoate, and polyethylene glycol diheptanoate, propylene glycol di(2-ethylhexanoate) and their mixtures, C₁₂-C₁₅ alcohol benzoates, hexyl laurate, esters of neopentanoic acid, such as isodecyl neopentanoate, isotridecyl neopentanoate, isostearyl neopentanoate or octyldodecyl neopentanoate, esters of isononanoic acid, such as isononyl isononanoate, isotridecyl isononanoate, octyl isononanoate, or hydroxylated esters, such as isostearyl lactate and diisostearyl malate.

According to an advantageous embodiment, the composition according to the invention comprises at least one branched oil ester.

“Branched oil ester” means one ester issued from the esterification of a polyol (having at least two OH functions) with a fatty acid.

More specifically, the ester oil is chosen from the octyldodecyl stearate and the branched esters such as polyesters of pentaerythrytol and carboxylic acid in C₄-C₄₀ such as pentaerythrityl tetraisostearate, dipentaerythrityl hexacaprylate/hexacaprate, pentaerythrityl isostearate, pentaerythrityl caprate, pentaerythrityl caprylate and their mixtures.

The mixture sold under the trademark “Supermol L®” from CRODA (mixtures of pentaerythrityl isosterate/caprylate/adipate) can be used.

Mention may also be made, by way of illustration of ester oils, of hydrocarbon oils of vegetable origin, such as phytosteryl esters, for example phytosteryl oleate, phytosteryl isostearate and lauroyl/octyldodecyl/phytosteryl glutamate (Ajinomoto, Eldew PS203), triglycerides composed of esters of fatty acids and of glycerol, the fatty acids of which in particular can have chain lengths varying from C₄-C₃₆ and in particular from C₁₈-C₃₆, it being possible for these oils to be linear or branched and saturated or unsaturated; these oils can in particular be heptanoic or octanoic triglycerides; wheat germ, sunflower, grape seed, sesame, maize, apricot, castor, shea, avocado, olive, soybean, sweet almond, palm, rapeseed, cottonseed, hazelnut, jojoba, alfalfa, poppy, pumpkinseed, cucumber, blackcurrant seed, evening primrose, millet, barley, guinea, rye, safflower, candlenut, passionflower and musk rose oils; shea butter; or triglycerides of caprylic/capric acids, such as those sold by Stéarineries Dubois or those sold under the names Miglyol 810®, 812® and 818® by Dynamit Nobel, and
esters of dimer diols and dimer diacids, such as Lusplan DD-DA5 and Lusplan DD-DA7®, sold by Nippon Fine Chemical and described in Application US 2004-175338, the content of which is incorporated in the present patent application by way of reference.

Among other ester oils convenient for the invention, the ester oils which are branched oils with a molecular weight of less than 4000 g/mol are more particularly distinguished, such as trimethylolpropane derivatives (ditrimethylolpropane tetraisostearate, trimethylolpropane triisostearate) and liquid triglycerides of fatty acids of 4 to 18 carbon atoms, such as castor oil, triglycerides of octanoic or heptanoic acids, or jojoba oil.

More particularly, a branched ester oil convenient for the invention can be chosen from pentaerythrityl isostearate, pentaerythrityl caprate, pentaerythrityl caprylate and their mixtures, and more particularly is pentaerythrityl isostearate.

As emerges from the above, the compositions of the invention comprise at least one fatty phase. Generally, this fatty phase is formulated in a physiologically acceptable medium.

Thus, the composition of the invention must be cosmetically or dermatologically acceptable, namely comprise a physiologically acceptable nontoxic medium capable of being applied to the lips of human beings. The term “cosmetically acceptable” is understood to mean, within the meaning of the invention, a composition with a pleasant appearance, pleasant smell and pleasant feel.

Fatty Phase

In addition to the abovementioned constituents, this fatty phase can comprise additional liquid or solid fatty substances, with the proviso that these are not prejudicial to the properties required according to the present invention.

The composition according to the invention can thus comprise additional nonvolatile oils other than the macadamia oil or derivatives and different form the previous ester oils, which can be chosen from hydrocarbon and/or silicone and/or fluorinated oils.

The term “hydrocarbon oil” is understood to mean an oil formed essentially, and even composed, of carbon and hydrogen atoms and optionally of oxygen or nitrogen atoms and not comprising a silicon or fluorine atom. It can comprise alcohol, ether, carboxylic acid, amine and/or amide groups.

The term “silicone oil” is understood to mean an oil comprising at least one silicon atom and in particular comprising Si—O groups.

These oils can be of vegetable, mineral or synthetic origin.

However, according to a preferred alternative form, the choice of additional oils is made out of a concern to respond to the requests of users for “natural” products or from natural origin. Consequently, the compositions according to the invention generally have a very small amount of silicone oil, indeed even are entirely devoid of it.

Mention may be made, by way of example of additional nonvolatile oil which can be used in the invention, of:
linear or branched hydrocarbons of mineral or synthetic origin, such as liquid paraffins and their derivatives, or liquid petrolatum;
fatty alcohols having from 12 to 26 carbon atoms, such as octyldodecanol, 2-butyloctanol, 2-hexyldecanol, 2-undecylpentadecanol and oleyl alcohol;
silicone oils, such as linear or cyclic polydimethylsiloxanes (PDMS); or polydimethylsiloxanes comprising pendent alkyl, alkoxy or phenyl groups or alkyl, alkoxy or phenyl groups at the silicone chain end, which groups have from 2 to 24 carbon atoms;
fatty acids having from 12 to 26 carbon atoms, such as oleic acid;
   and their mixtures.

The additional nonvolatile oil can be present in a content ranging from 0.1 to 80% by weight, preferably from 1 to 60% by weight, better still from 5 to 50% by weight, and even better still from 5 to 40% by weight, with respect to the total weight of the composition.

Preferably, a composition according to the invention exhibits a low content of volatile oil, that is to say a content of less than or equal to 10% by weight, with respect to the total weight of the composition.

Thus, according to one embodiment, a composition of the invention comprises less than 2%, indeed even less than 1%, of volatile oil or else is completely devoid of volatile oil.

The term “volatile oil” is understood to mean, within the meaning of the invention, an oil capable of evaporating on contact with keratinous substances in less than one hour, at ambient temperature and atmospheric pressure. The volatile organic solvent or solvents and the volatile oils of the invention are liquid at ambient temperature and have a non-zero vapour pressure, at ambient temperature and at atmospheric pressure, ranging in particular from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

These oils can be hydrocarbon oils, silicone oils, fluorinated oils or their mixtures.

Volatile hydrocarbon oils can be chosen from hydrocarbon oils having from 8 to 16 carbon atoms and in particular branched C₈-C₁₆ alkanes, such as C₈-C₁₆ isoalkanes of petroleum origin (also known as isoparaffins), such as isododecane (also known as 2,2,4,4,6-pentamethylheptane), isodecane, isohexadecane and, for example, the oils sold under the trade names of Isopars or Permethyls, branched C₈-C₁₆ esters, isohexyl neopentanoate and their mixtures. Other volatile hydrocarbon oils, such as petroleum distillates, in particular those sold under the name Shell Solt by Shell, can also be used. Preferably, the volatile solvent is chosen from volatile hydrocarbon oils having from 8 to 16 carbon atoms and their mixtures.

Use may also be made, as volatile oils, of volatile silicones, such as, for example, volatile linear or cyclic silicone oils, in particular those having a viscosity ≦8 centistokes (8×10⁻⁶ m²/s) and having in particular from 2 to 7 silicon atoms, these silicones optionally comprising alkyl or alkoxy groups having from 1 to 10 carbon atoms. Mention may in particular be made, as volatile silicone oil which can be used in the invention, of octamethylcyclotetrasiloxane, decamethylcyclopentasiloxane, dodecamethyl-cyclohexasiloxane, heptamethylhexyltrisiloxane, heptamethyloctyltrisiloxane, hexamethyl-disiloxane, octamethyltrisiloxane, decamethyltetrasiloxane, dodecamethylpentasiloxane and their mixtures.

Likewise, a composition can comprise, in addition to a wax of natural origin, other additional waxes as defined below or pasty compounds.

Additional Waxes

A composition of the invention can comprise at least one additional wax.

The additional waxes capable of being used in the compositions according to the invention, in combination with at least one polyglycerolated beeswax, are chosen from other wax from natural origine, synthetic waxes which are solid at ambient temperature and their mixtures.

Advantageously, the associated wax or waxes can be chosen from nonpolar waxes and ester waxes.

The term “ester wax” is understood to mean, according to the invention, a wax comprising at least one ester functional group.

Use may in particular be made, as ester wax, of the waxes obtained by hydrogenation of castor oil esterified with cetyl alcohol sold under the names of Phytowax castor 16L64® and 22L73® by Sophim. Such waxes are described in Application FR-A-2 792 190.

Mention may also be made, as ester wax which can be used, of di(1,1,1-trimethylolpropane) tetrastearate, sold under the name of Hest 2T-4S® by Heterene.

Also to be understood as ester wax are esters of formula R₁COOR₂ in which R₁ and R₂ represent linear, branched or cyclic aliphatic chains, the number of atoms of which varies from 10 to 50, which can comprise a heteroatom, such as O, N or P, and which have a melting point temperature varying from 25 to 120° C.

According to a preferred embodiment, the composition of the invention comprises at least one additional wax and in particular at least one nonpolar wax.

The nonpolar waxes are in particular hydrocarbon waxes composed solely of carbon and hydrogen atoms and devoid of heteroatoms, such as N, O, Si and P.

Mention may in particular be made, by way of illustration of the nonpolar waxes suitable for the invention, of hydrocarbon waxes, such as microcrystalline waxes, paraffin waxes, ozokerite and polyethylene waxes.

Mention may be made, as ozokerite, of Ozokerite Wax SP 1020 P.

Mention may be made, as microcrystalline waxes which can be used, of Multiwax W 445®, sold by Sonneborn and Microwax HW® and Base Wax 30540®, sold by Paramelt.

According to a preferred embodiment, the composition of the invention comprises at least one further wax, chosen among the nonpolar wax and more preferably such nonpolar wax is a microcrystalline wax, in particular like MICROWAX HW® from PARAMELT.

The presence of at least one nonpolar wax such as a microcrystalline wax in the composition of the invention allows in particular to increase its hardness.

Preferably, the composition can comprise a content of nonpolar wax ranging from 3 to 18% by weight; in particular, it can comprise from 5 to 13% by weight thereof, with respect to the total weight of the composition.

This additional wax may in particular be an wax from natural origin.

Within the meaning of the present invention, a “wax of natural origin” implies that the wax is not a synthetic wax, that is to say is not obtained by chemical synthesis.

It results from a vegetable, animal or mineral source.

More specifically, the wax of natural origin present in the compositions of the invention can be an animal or vegetable wax.

However, this natural wax may have been subjected to additional treatments of refining and/or purifying type or also to a chemical reaction, such as esterification or etherification, for example. These modified waxes of natural origin are also suitable for the present invention and are covered by the definition “wax of natural origin”.

Mention may in particular be made, by way of illustration of the waxes of natural origin suitable for the invention, of hydrocarbon waxes of natural origin, lanolin wax, Chinese insect waxes, rice bran wax, carnauba wax, candelilla wax, ouricury wax, esparto wax, berry wax, shellac wax, Japan wax, sumac wax, montan wax, orange and lemon waxes, and their derived products.

Mention may also be made of waxes obtained by catalytic hydrogenation of animal or vegetable oils having linear or branched C₈-C₃₂ fatty chains. Mention may in particular be made, among these, of isomerized jojoba oil, such as the trans-isomerized partially hydrogenated jojoba oil manufactured or sold by Desert Whale under the commercial reference Iso-Jojoba-50®, hydrogenated sunflower oil, hydrogenated castor oil, hydrogenated coconut oil or hydrogenated lanolin oil.

A composition according to the invention can comprise a total content of additional waxes ranging from 0.5 to 25% by weight, in particular ranging from 2 to 20% by weight and especially ranging from 5 to 15% by weight, with respect to the total weight of the composition.

Preferably, a composition can comprise a total content of wax ranging from 2 to 15% by weight, with respect to the total weight of the composition; in particular, it can comprise from 5 to 15% by weight thereof.

Preferably, when a composition according to the invention is in a solid form, this comprises a total content of waxes ranging from 8 to 15%.

Pasty Compounds

The composition according to the invention can comprise, in addition to the waxes, another solid fatty substance, such as at least one pasty compound.

The term “pasty” is understood to mean, within the meaning of the present invention, a lipophilic fatty compound with a reversible solid/liquid change in state which exhibits, in the solid state, an anisotropic crystalline arrangement and which comprises, at a temperature of 23° C., a liquid fraction and a solid fraction.

In other words, the starting melting point of the pasty compound can be less than 23° C. The liquid fraction of the pasty compound, measured at 23° C., can represent from 9 to 97% by weight of the compound. At 23° C., this liquid fraction preferably represents between 15 and 85% by weight, more preferably between 40 and 85% by weight.

The liquid fraction by weight of the pasty compound at 23° C. is equal to the ratio of the enthalpy of fusion consumed at 23° C. to the enthalpy of fusion of the pasty compound.

The enthalpy of fusion of the pasty compound is the enthalpy consumed by the compound to change from a solid state to liquid state. The pasty compound is “in the solid state” when the whole of its mass is in the solid form. The pasty compound is “in the liquid state” when the whole of this mass is in the liquid form.

The enthalpy of fusion of the pasty compound is equal to the area under the curve of the thermogram obtained using a differential scanning calorimeter, such as the calorimeter sold under the name MDSC 2920 by TA Instruments, with a rise in temperature of 5 or 10° C. per minute, according to Standard ISO 11357-3:1999. The enthalpy of fusion of the pasty compound is the amount of energy necessary to change the compound from the solid state to the liquid state. It is expressed in J/g.

The enthalpy of fusion consumed at 23° C. is the amount of energy absorbed by the sample to change from the solid state to the state which it exhibits at 23° C., composed of a liquid fraction and of a solid fraction.

The liquid fraction of the pasty compound measured at 32° C. preferably represents from 30 to 100% by weight of the compound, preferably from 50 to 100% by weight of the compound, more preferably from 60 to 100% by weight of the compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the enthalpy of fusion consumed at 32° C. to the enthalpy of fusion of the pasty compound. The enthalpy of fusion consumed at 32° C. is calculated in the same way as the enthalpy of fusion consumed at 23° C.

The pasty compound can advantageously be chosen from:
lanolin and its derivatives
polymeric or nonpolymeric silicone compounds,
polymeric or nonpolymeric fluorinated compounds,
vinyl polymers, in particular:
   olefin homopolymers,
   olefin copolymers,
   hydrogenated diene homopolymers and copolymers,
   linear or branched and homo- or copolymeric oligomers of alkyl (meth)acrylates preferably having a C₈-C₃₀ alkyl group,
   homo- and copolymeric oligomers of vinyl esters having C₈-C₃₀ alkyl groups,
   homo- and copolymeric oligomers of vinyl ethers having C₈-C₃₀ alkyl groups,
fat-soluble polyethers resulting from polyetherification between one or more C₂-C₁₀₀, preferably C₂-C₅₀, diols,
esters,
and their mixtures.

The pasty compound is preferably chosen from synthetic compounds and compounds of vegetable origin. A pasty compound can be obtained by synthesis starting from starting materials of vegetable origin.

Preference is in particular given, among esters, to:
the esters of an oligomeric glycerol, in particular the esters of diglycerol, especially the condensates of adipic acid and of glycerol, for which a portion of the hydroxyl groups of the glycerols have reacted with a mixture of fatty acids, such as stearic acid, capric acid, stearic acid and isostearic acid and 12-hydroxystearic acid, such as, in particular, those sold under the Softisan 649 brand by Sasol,
the arachidyl propionate sold under the Waxenol 801 brand by Alzo,
phytosterol esters,
triglycerides of fatty acids and their derivatives,
pentaerythritol esters,
noncrosslinked polyesters resulting from the polycondensation between a linear or branched C₄-C₅₀ dicarboxylic acid or polycarboxylic acid and a C₂-C₅₀ diol or polyol, like for example Elfacos ST9 d'AKZO NOBEL,
aliphatic esters of an ester resulting from the esterification of an aliphatic hydroxycarboxylic acid ester by an aliphatic carboxylic acid,
polyesters resulting from the esterification, by a polycarboxylic acid, of an aliphatic hydroxycarboxylic acid ester, the said ester comprising at least two hydroxyl groups, such as the products Risocast DA-H® and Risocast DA-L®,
dimer diol and dimer diacid esters, if appropriate esterified on their free alcohol or acid functional group(s) by acid or alcohol radicals, such as Plandool-G,
and their mixtures.

The choice will preferably be made, among pasty compounds of vegetable origin, of a mixture of soya sterols and of oxyethylenated (5 EO)/oxypropylenated (5 PO) pentaerythritol sold under the reference Lanolide by Vevy.

A composition can comprise a total content of pasty fatty substances ranging from 0.5 to 50% by weight, preferably ranging from 1 to 40% by weight and better still ranging from 5 to 30% by weight, with respect to the weight of the composition.

The composition according to the invention can comprise, in addition to the waxes optionally present, at least one structuring agent chosen from semicrystalline polymers, lipophilic gelling agents and their mixtures.

Thus, according to one embodiment, the composition according to the invention can comprise at least one gelling agent. The gelling agents which can be used in the compositions according to the invention can be polymeric or molecular and organic or inorganic lipophilic gelling agents.

Mention may be made, as inorganic lipophilic gelling agent, of optionally modified clays, such as hectorites modified by a C₁₀-C₂₂ ammonium chloride, such as hectorite modified by distearyldimethylammonium chloride, such as, for example, that sold under the name of Bentone 38V® by Elementis.

Mention may also be made of pyrogenic silica optionally treated hydrophobically at the surface, the size of the particles of which is less than 1 μm. This is because it is possible to chemically modify the surface of the silica by chemical reaction, resulting in a reduction in the number of silanol groups present at the surface of the silica. It is possible in particular to substitute silanol groups by hydrophobic groups: a hydrophobic silica is then obtained. The hydrophobic groups can be:
trimethylsiloxyl groups, which are obtained in particular by treatment of pyrogenic silica in the presence of hexamethyldisilazane. Silicas thus treated are named “Silica silylate” according to the CTFA (8th edition, 2000). They are, for example, sold under the references Aerosil R812® by Degussa and Cab-O—Sil TS-530® by Cabot,
dimethylsilyloxyl or polydimethylsiloxane groups, which are obtained in particular by treatment of pyrogenic silica in the presence of polydimethylsiloxane or dimethyldichlorosilane. Silicas thus treated are named “Silica dimethyl silylate” according to the CTFA (8th edition, 2000). They are, for example, sold under the references Aerosil R972® and Aerosil R974® by Degussa and Cab-O-Sil TS-610® and Cab-O-Sil TS-720® by Cabot.

The hydrophobic pyrogenic silica exhibits in particular a particle size which can be nanometric to micrometric, for example ranging from approximately 5 to 200 nm.

The polymeric organic lipophilic gelling agents are, for example, partially or completely crosslinked organopolysiloxane elastomers of three-dimensional structure, such as those sold under the names of KSG6®, KSG16® and KSG18 by Shin-Etsu, of Trefil E-505C® and Trefil E-506C® by Dow Corning, of Gransil SR-CYC®, SR DMF10®, SR-DC556®, SR 5CYC Gel®, SR DMF 10 Gel® and SR DC 556 Gel® by Grant Industries, of SF 1204® and of JK 113® by General Electric; ethylcellulose, such as that sold under the name Ethocel® by Dow Chemical; galactomannans comprising from one to six and in particular from two to four hydroxyl groups per monosaccharide which are substituted by a saturated or unsaturated alkyl chain, such as guar gum alkylated by C₁ to C₆ alkyl chains, in particular C₁ to C₃ alkyl chains, and their mixtures; block copolymers of “diblock”, “triblock” or “radial” type of the polystyrene/polyisoprene or polystyrene/polybutadiene type, such as those sold under the name Luvitol HSB® by BASF, of the polystyrene/copoly(ethylene-propylene) type, such as those sold under the name Kraton® by Shell Chemical Co., or of the polystyrene/copoly(ethylene-butylene) type, blends of triblock and radial (star) copolymers in isododecane, such as those sold by Penreco under the name Versagel®, such as, for example, the blend of butylene/ethylene/styrene triblock copolymer and of ethylene/propylene/styrene star copolymer in isododecane (Versagel M 5960).

Mention may also be made, as lipophilic gelling agent, of polymers with a weight-average molecular weight of less than 100 000 comprising a) a polymer backbone having hydrocarbon repeat units provided with at least one heteroatom and optionally b) at least one pendent fatty chain and/or at least one end fatty chain which are optionally functionalized, having from 6 to 120 carbon atoms and being bonded to these hydrocarbon units, such as described in Applications WO-A-02/056847 and WO-A-02/47619, the contents of which are incorporated by way of reference; in particular, polyamide resins (in particular comprising alkyl groups having from 12 to 22 carbon atoms), such as those described in U.S. Pat. No. 5,783,657, the content of which is incorporated by way of reference.

Mention may also be made, among the lipophilic gelling agents which can be used in the compositions according to the invention, of esters of dextrin and of fatty acid, such as dextrin palmitates, in particular such as those sold under the names Rheopearl TL® or Rheopearl KL® by Chiba Flour.

Use may also be made of silicone polyamides of the polyorganosiloxane type, such as those described in the documents U.S. Pat. No. 5,874,069, U.S. Pat. No. 5,919,441, U.S. Pat. No. 6,051,216 and U.S. Pat. No. 5,981,680.

These silicone polymers can belong to the following two families:
polyorganosiloxanes comprising at least two groups capable of establishing hydrogen interactions, these two groups being situated in the chain of a polymer, and/or
polyorganosiloxanes comprising at least two groups capable of establishing hydrogen interactions, these two groups being situated on grafts or branchings.

Film-Forming Agent

The compositions can also comprise at least one film-forming polymer.

According to one embodiment, the composition comprises at least one polymer which can be chosen from film-forming polymers.

The term “polymer” is to be understood to mean, within the meaning of the invention, a compound having at least two repeat units and preferably at least three repeat units.

The term “film-forming polymer” is to be understood to mean a polymer capable of forming, by itself alone or in the presence of an additional agent which is able to form a film, a macroscopically continuous film on a support, in particular on keratinous substances.

The polymer can be present in the composition in a content ranging from 0.1% to 60% by weight, preferably ranging from 0.1% to 50% by weight, preferably ranging from 0.5 to 40% by weight, preferentially ranging from 1% to 30% by weight and more preferentially ranging from 1% to 25% by weight, with respect to the total weight of the composition.

In one embodiment, the film-forming organic polymer is at least a polymer chosen from the group consisting of:
film-forming polymers which are soluble in an organic liquid medium, in particular fat-soluble polymers, when the organic liquid medium comprises at least one oil;
film-forming polymers which are dispersible in an organic solvent medium, in particular polymers in the form of nonaqueous dispersions of polymer particles, preferably dispersions in silicone or hydrocarbon oils.

According to one embodiment, the nonaqueous dispersions of polymers comprise polymer particles stabilized on their surface by at least one stabilizing agent.
film-forming polymers in the form of aqueous dispersions of polymer particles, often known as “latexes”; in this case, the composition comprises an aqueous phase;
water-soluble film-forming polymers; in this case, the composition comprises an aqueous phase.

Mention may be made, among film-forming polymers which can be used in the composition of the present invention, of synthetic polymers of radical type or of polycondensate type, polymers of natural origin and their blends. Mention may in particular be made, as film-forming polymer, of acrylic polymers, polyurethanes, polyesters, polyamides, polyureas, silicone polymers, silicone-grafted acrylic polymers, polyamide polymers and copolymers and polyisoprenes.

It is understood that the amount of these additional compounds can be adjusted by a person skilled in the art so as not to prejudice the effect desired in the context of the present invention.

In addition to the abovementioned compounds, the composition according to the invention can comprise the compounds normally employed in the formulation of cosmetic compositions devoted to making up and/or caring for the lips.

Thus, conventionally, the cosmetic compositions according to the invention can comprise fillers and/or colouring materials.

Colouring Materials

The composition according to the invention can in addition comprise one or more colouring materials chosen from water-soluble dyes and pulverulent colouring materials, such as pigments, pearlescent agents and glitter, well known to a person skilled in the art.

The colouring materials can be present in the composition in a proportion of 0.01% to 40% by weight, in particular of 0.01% to 30% by weight and especially of 0.05 to 25% by weight, with respect to the total weight of the composition.

The term “pigments” should be understood as meaning white or coloured and inorganic or organic particles which are insoluble in an aqueous solution and which are intended to colour and/or opacify the resulting film.

The pigments can be present in a proportion of 0.01 to 20% by weight, in particular of 0.01 to 15% by weight and especially of 0.02 to 10% by weight, with respect to the total weight of the cosmetic composition.

Mention may be made, as inorganic pigments which can be used in the invention, of titanium, zirconium or cerium oxides, and also of zinc, iron or chromium oxides, ferric blue, manganese violet, ultramarine blue and chromium hydrate.

The pigment can also have a structure which can, for example, be of sericite/brown iron oxide/titanium dioxide/silica type. Such a pigment is sold, for example, under the reference Coverleaf NS or JS by Chemicals and Catalysts and exhibits a contrast ratio in the region of 30.

The colouring material can also comprise a pigment having a structure which can, for example, be of the type of silica microspheres comprising iron oxide. An example of the pigment exhibiting this structure is that sold by Miyoshi under the reference PC Ball PC-LL-100 P, this pigment being composed of silica microspheres comprising yellow iron oxide.

Mention may be made, among the organic pigments which can be used in the invention, of carbon black, pigments of D & C type, lakes based on cochineal carmine of barium, strontium, calcium or aluminium, or the diketopyrrolopyrroles (DPP) disclosed in the documents EP-A-542 669, EP-A-787 730, EP-A-787 731 and WO-A-96/08537.

The term “pearlescent agents” should be understood as meaning coloured particles of any shape, which may or may not be iridescent, produced in particular by certain shellfish in their shells or synthesized, which exhibit a colouring effect by optical interference.

The pearlescent agents can be chosen from pearlescent pigments, such as titanium oxide-coated mica covered with iron oxide, with bismuth oxychloride, with chromium oxide or with an organic dye, and pearlescent pigments based on bismuth oxychloride. They can also be mica particles, at the surface of which at least two successive layers of metal oxides and/or of organic colouring materials are superimposed.

Mention may also be made, as examples of pearlescent agents, of natural mica covered with titanium oxide, with iron oxide, with natural pigment or with bismuth oxychloride.

Mention may be made, among the pearlescent agents available on the market, of the Timica, Flamenco and Duochrome (mica-based) pearlescent agents sold by Engelhard, the Timiron pearlescent agents sold by Merck, the Prestige mica-based pearlescent agents sold by Eckart and the Sunshine synthetic mica-based pearlescent agents sold by Sun Chemical.

The pearlescent agents can more particularly have a yellow, pink, red, bronze, orangey, brown, gold and/or coppery colour or glint.

Mention may in particular be made, by way of illustration of the pearlescent agents which can be employed in the context of the present invention, of pearlescent agents of gold colour sold in particular by Engelhard under the name of Brilliant Gold 212G (Timica), Gold 222C (Cloisonne), Sparkle Gold (Timica), Gold 4504 (Chromalite) and Monarch Gold 233X (Cloisonne); bronze pearlescent agents sold in particular by Merck under the names Bronze Fine (17384) (Colorona) and Bronze (17353) (Colorona) and by Engelhard under the name Super Bronze (Cloisonne); orange pearlescent agents sold in particular by Engelhard under the names Orange 363C (Cloisonne) and Orange MCR 101 (Cosmica) and by Merck under the names Passion Orange (Colorona) and Matte Orange (17449) (Microna); brown-coloured pearlescent agents sold in particular by Engelhard under the names Nu Antique Copper 340XB (Cloisonne) and Brown CL4509 (Chromalite); pearlescent agents with a copper glint sold in particular by Engelhard under the name Copper 340A (Timica); pearlescent agents with a red glint sold in particular by Merck under the name Sienna Fine (17386) (Colorona); pearlescent agents with a yellow glint sold in particular by Engelhard under the name Yellow (4502) (Chromalite); red-coloured pearlescent agents with a gold glint sold in particular by Engelhard under the name Sunstone G012 (Gemtone); pink pearlescent agents sold in particular by Engelhard under the name Tan Opale G005 (Gemtone); black pearlescent agents with a gold glint sold in particular by Engelhard under the name Nu Antique Bronze 240 AB (Tunica); blue pearlescent agents sold in particular by Merck under the name Matte Blue (17433) (Microna); white pearlescent agents with a silvery glint sold in particular by Merck under the name Xirona Silver; and golden green pinkish orangey pearlescent agents sold in particular by Merck under the name Indian Summer (Xirona); and their mixtures.

The term “dyes” should be understood as meaning compounds, generally organic compounds, which are soluble in fatty substances, such as oils, or in an aqueous/alcoholic phase.

The fat-soluble dyes can be chosen from Sudan red, DC Red 17, DC Green 6, β-carotene, Sudan brown, DC Yellow 11, DC Violet 2, DC Orange 5 and quinoline yellow. The water-soluble dyes are, for example, beetroot juice and methylene blue.

According to a specific embodiment, the colouring material(s) present in the compositions according to the invention is (are) chosen from pigments, pearlescent agents, water-soluble or fat-soluble dyes and their mixtures.

The cosmetic composition according to the invention can also comprise at least one material with a specific optical effect.

This effect is different from a simple conventional colouring effect, that is to say a unified and stabilized effect such as produced by conventional colouring materials, such as, for example, monochromatic pigments. Within the meaning of the invention, the term “stabilized” means devoid of an effect of variability in the colour with the angle of observation or else in response to a change in temperature.

For example, this material can be chosen from particles with a metallic glint, goniochromatic colouring agents, diffracting pigments, thermochromic agents, optical brighteners and fibres, in particular interference fibres. Of course, these various materials can be combined so as to provide the simultaneous display of two effects, indeed even of a novel effect in accordance with the invention.

The particles with a metallic glint which can be used in the invention are chosen in particular from:
particles of at least one metal and/or of at least one metal derivative,
particles comprising an organic or inorganic substrate, made of one or more materials, at least partially covered with at least one layer with a metallic glint comprising at least one metal and/or at least one metal derivative, and
mixtures of the said particles.

Mention may be made, among the metals which can be present in the said particles, for example, of Ag, Au, Cu, Al, Ni, Sn, Mg, Cr, Mo, Ti, Zr, Pt, Va, Rb, W, Zn, Ge, Te, Se and their mixtures or alloys. Ag, Au, Cu, Al, Zn, Ni, Mo, Cr and their mixtures or alloys (for example, bronzes and brasses) are preferred metals.

The term “metal derivatives” denotes compounds derived from metals, in particular oxides, fluorides, chlorides and sulphides.

Mention may be made, by way of illustration of these particles, of aluminium particles, such as those sold under the names Starbrite 1200 EAC® by Siberline and Metalure® by Eckart.

Mention may also be made of metal powders formed of copper or alloy mixtures, such as the references 2844 sold by Radium Bronze, metal pigments, such as aluminium or bronze, for example those sold under the names Rotosafe 700 from Eckhart, silica-coated aluminium particles sold under the name Visionaire Bright Silver from Eckhart and particles formed of metal alloy, such as powders formed of bronze (copper and zinc alloy) coated with silica sold under the name Visionaire Bright Natural Gold from Eckart.

The particles can also comprise a glass substrate, such as those sold by Nippon Sheet Glass under the names Microglass Metashine.

The goniochromatic colouring agent can be chosen, for example, from interference multilayered structures and liquid crystal colouring agents.

Examples of symmetrical interference multilayer structures which can be used in compositions produced in accordance with the invention are, for example, the following structures: Al/SiO₂/Al/SiO₂/Al, pigments having this structure being sold by DuPont de Nemours; Cr/MgF₂/Al/MgF₂/Cr, pigments having this structure being sold under the name Chromaflair by Flex; MoS₂/SiO₂/Al/SiO₂/MoS₂; Fe₂O₃/SiO₂/Al/SiO₂/Fe₂O₃ and Fe₂O₃/SiO₂/Fe₂O₃/SiO₂/Fe₂O₃, pigments having these structures being sold under the name Sicopearl by BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona by Merck (Darmstadt). By way of examples, these pigments can be pigments with a silica/titanium oxide/tin oxide structure sold under the name Xirona Magic by Merck, pigments with a silica/brown iron oxide structure sold under the name Xirona Indian Summer by Merck and pigments with a silica/titanium oxide/mica/tin oxide structure sold under the name Xirona Caribbean Blue by Merck. Mention may also be made of the Infinite Colors pigments from Shiseido. Different effects are obtained according to the thickness and the nature of the various layers. Thus, with the structure Fe₂O₃/SiO₂/Al/SiO₂/Fe₂O₃, the colour changes from green-golden to red-grey for SiO₂ layers of 320 to 350 nm; from red to golden for SiO₂ layers of 380 to 400 nm; from purple to green for SiO₂ layers of 410 to 420 nm; and from copper to red for SiO₂ layers of 430 to 440 nm.

Mention may be made, as examples of pigments with a polymeric multilayer structure, of those sold by 3M under the name Color Glitter®.

Use may be made, as liquid crystal goniochromatic particles, for example, of those sold by Chenix and of that sold under the name Helicone® HC by Wacker.

Filler

Advantageously, a composition according to the invention comprises at least one filler, in particular in a total content ranging from 0.1% to 30% by weight, preferably ranging from 0.1% to 15% by weight and preferentially ranging from 0.5% to 10% by weight, with respect to the total weight of the composition.

The term “fillers” should be understood as meaning, within the meaning of the present invention, colourless or white and inorganic or synthetic particles of any shape which are insoluble in the medium of the composition, whatever the temperature at which the composition is manufactured. These fillers serve in particular to modify the rheology or the texture of the composition.

The fillers can be inorganic or organic and of any shape, platelet, spherical or oblong, whatever the crystallographic form (for example sheet, cubic, hexagonal, orthorhombic, and the like). Mention may be made of talc, mica, silica, kaolin, powders formed of polyamide (Nylon®) (Orgasol® from Atochem), of poly-β-alanine and of polyethylene, powders formed of tetrafluoroethylene polymers (Teflon®), lauroyllysine, starch, boron nitride, hollow polymeric microspheres, such as those of poly(vinylidene chloride)/acrylonitrile, for example Expancel® (Nobel Industrie), or of acrylic acid copolymers (Polytrap® from Dow Corning), silicone resin microbeads (Tospearls® from Toshiba, for example), polyorganosiloxane elastomer particles, precipitated calcium carbonate, magnesium carbonate, basic magnesium carbonate, hydroxyapatite, hollow silica microspheres (Silica Beads® from Maprecos), glass or ceramic microcapsules, or metal soaps derived from organic carboxylic acids having from 8 to 22 carbon atoms, preferably from 12 to 18 carbon atoms, for example zinc stearate, magnesium stearate, lithium stearate, zinc laurate or magnesium myristate.

The fillers can also be particles comprising a copolymer, the said copolymer comprising trimethylol hexyllactone. In particular, a hexamethylene diisocyanate/trimethylol hexyllactone copolymer may be involved. Such particles are in particular available commercially, for example under the name of Plastic Powder D-400® or Plastic Powder D-800® from Toshiki.

Additional Normal Cosmetic Ingredients

The composition according to the invention can additionally comprise any known cosmetic ingredient which can be chosen in particular from antioxidants, fragrances, preservatives, neutralizing agents, surfactants, sunscreens, vitamins, moisturizing agents, emollients, hydrophilic or lipophilic active principles, agents for combating free radicals, sequestering agents and their mixtures.

Of course, a person skilled in the art will take care to choose the optional additional ingredients and/or their amounts so that the advantageous properties of the composition according to the invention are not substantially detrimentally affected by the envisaged addition.

A composition according to the invention can be manufactured by the known processes generally used in the cosmetic or dermatological field.

For example, it can be manufactured by the following process.

In a first step, the pigments can be milled in a portion of the oily phase. The remaining fat-soluble ingredients can subsequently be mixed at a temperature of the order of 100° C. The milled material or the predispersed active principles can then be added to the oily phase. The optional hydrophilic active principles can subsequently be dispersed using a mechanical stirrer. The pearlescent agents and the fillers are introduced at the end of the process.

Finally, in the case of a stick, the composition can be cast in a mould capable of providing it with the final shape and everything can be left to cool at ambient temperature and/or in a freezer.

The invention is illustrated in more detail in the following example, given by way of illustration and without a limiting nature. The percentages are percentages expressed by weight.

### EXAMPLE 1

A lipstick formulation is prepared:

| | INCI | % by |
|---|---|---|
| INGREDIENTS | COMMERCIAL REFERENCE | weight |
| PHASE A | Pentaerythrityl isostearate/caprate/caprylate | 21.7 |
| | mixture | |
| | Supermol L ® from Croda | |
| | Macadamia ternifolia seed oil | 18.7 |
| | Macadamia oil of C ® type from | |
| | Nestlé World Trade Corporation | |
| | Octyldodecyl stearate | 18.7 |
| | Lexiol 3820 from Cognis | |
| | Bis-diglyceryl polyacyladipate-2 | 2.7 |
| | Softisan 649 ® from Sasol | |
| | PEG-45/dodecyl glycol copolymer | 7.4 |
| | Elfacos ST 9 ® from Akzo Nobel | |
| | Hydrogenated castor oil dimer dilinoleate | 2.7 |
| | Risocast DA-L ® from Kokyu Alcohol | |
| | Kogyo | |
| | Pigments | 5 |
| SUNSCREEN | Ethylhexyl methoxycinnamate | 3.7 |
| | Uvinul MC 80 ® from BASF | |
| FRAGRANCE | Fragrance | 0.4 |
| WAX | Polyglyceryl-3 beeswax | 7.5 |
| | Cera Bellina ® wax from Koster Keunen | |
| | Microcrystalline wax | 7.6 |
| | Microwax HW ® from Paramelt | |
| PEARLESCENT | Mica (and) iron oxides (and) titanium oxides | 3.9 |
| AGENT | | |

It is obtained according to the following protocol:

In a first step, the pigments are milled in a portion of phase A. The remaining fat-soluble ingredients are subsequently mixed at a temperature of the order of 100° C. The milled material is then added. The screening agent, the pearlescent agents and the fragrance are subsequently added.

Finally, the composition is cast in a mould which makes it possible to obtain a stick with a diameter of 12.7 mm and everything is left to cool in a freezer for approximately one hour.

It is confirmed that the composition according to the invention is soft and melting on application.

The film applied on the lips stays comfortable overtime and provides an efficient hydration to the lips.

Furthermore, the obtained sticks do not exhibit a phenomenon of recrystallisation or exudation overtime.

1.-17. (canceled)

18. An anhydrous cosmetic composition for making up and/or caring for the lips comprising at least one fatty phase comprising at least 0.5% by weight, with respect to the total weight of the composition, of at least one macadamia oil or one of its derivatives and at least one polyglycerolated beeswax.

19. The composition according to claim 18, comprising at least 2% by weight of macadamia oil or derivatives, with respect to the total weight of the composition.

20. The composition according to claim 18, wherein the polyglycerolated beeswax is the polyglycerol-3-beeswax.

21. The composition according to claim 18, comprising between 2% to 25% by weight of wax(es) of polyglycerolated beeswax, with respect to the weight of the fatty phase of the composition.

22. The composition according to claim 18, comprising at least one additional ester oil.

23. The composition according to claim 22, wherein the additional ester oil is selected from the group consisting of linear ester oils and branched ester oils.

24. The composition according to claim 18, additionally comprising at least one the linear ester of alcohol and of fatty acid.

25. The composition according to claim 24, wherein the linear ester of alcohol and of fatty acid is selected from the group consisting of cetostearyl octanoate, esters of isopropyl alcohol, ethyl palmitate, 2-ethylhexyl palmitate, isopropyl stearate or isostearate, isostearyl isostearate, octyl stearate, octyldodecyl stearate, pentaerythrityl isostearate or caprate or caprylate or their mixtures, hydroxylated esters, diisopropyl adipate, heptanoates octanoates, decanoates or ricinoleates of alcohols or of polyalcohols, alkyl benzoate, polyethylene glycol diheptanoate, propylene glycol di(2-ethylhexanoate) and their mixtures, C₁₂-C₁₅ alcohol benzoates, hexyl laurate, esters of neopentanoic acid, esters of isononanoic acid, and hydroxylated esters.

26. The composition according to claim 23, wherein the additional ester oil is selected from the group consisting of linear ester oils and branched ester oils.

27. The composition according to claim 18, comprising at least 5% by weight of ester oil(s), with respect to the total weight of the composition.

28. The composition according to claim 18, comprising at least one additional ester wax and/or one nonpolar wax.

29. The composition according to claim 28, wherein said nonpolar wax is a hydrocarbon wax.

30. The composition according to claim 18, comprising less than 4% by weight and of water, with respect to the total weight of the composition.

31. The composition according to claim 18, comprising at least one filler.

32. The composition according to claim 18, comprising at least one pasty compound.

33. The composition according to claim 18, comprising at least one colouring material selected from the group consisting of pigments, pearlescent agents, water-soluble or fat-soluble dyes and their mixtures.

34. The composition according to claim 18, in the solid form.

35. The composition according to claim 34, exhibiting a hardness varying from 30 to 400 g.

36. Method for making up or caring for the lips, comprising at least one stage which consists in applying, to the said lips, a composition as defined in claim 18.

37. The composition according to claim 25, wherein the linear ester of alcohol and of fatty acid is selected from the group consisting of cetostearyl octanoate, isopropyl myristate or isopropyl palmitate, ethyl palmitate, 2-ethylhexyl palmitate, isopropyl stearate or isostearate, isostearyl isostearate, octyl stearate, octyldodecyl stearate, pentaerythrityl isostearate or caprate or caprylate or their mixtures, isostearyl lactate, octyl hydroxystearate, diisopropyl adipate, propylene glycol dioctanoate, cetyl octanoate, tridecyl octanoate, 2-ethylhexyl palmitate, 4-diheptanoate, alkyl benzoate, polyethylene glycol diheptanoate, propylene glycol di(2-ethylhexanoate) and their mixtures, C₁₂-C₁₅ alcohol benzoates, hexyl laurate, isodecyl neopentanoate, isotridecyl neopentanoate, isostearyl neopentanoate, octyldodecyl neopentanoate, isononyl isononanoate, isotridecyl isononanoate, octyl isononanoate, isostearyl lactate, and diisostearyl malate.

38. The composition according to claim 26, wherein the additional ester oil is selected from the group consisting of octyldodecyl stearate, polyesters of pentaerythrytol and C₄-C₄₀ carboxylic acid, and their mixtures.

39. The composition according to claim 26, wherein the additional ester oil is selected from the group consisting of pentaerythrityl tetraisostearate, dipentaerythrityl hexacaprylate/hexacaprate, pentaerythrityl isostearate, pentaerythrityl caprate, pentaerythrityl caprylate, and their mixtures.
